(19) United States
(12) Patent Application Publication
(10) Pub. No.: US 2010/0178712 A1
(43) Pub. Date: Jul. 15, 2010

(54) Microplates containing microsphere fluorescence standards, microsphere standards, and methods for their use

(75) Inventor: Yu-Zhong Zhang, Eugene, OR (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/639,578

(22) Filed: Dec. 16, 2009

Related U.S. Application Data
(63) Continuation of application No. 11/218,032, filed on Aug. 31, 2005, now abandoned.
(60) Provisional application No. 60/606,694, filed on Sep. 1, 2004.

(51) Int. Cl. G01N 33/543; B01L 9/00; B05D 3/00; G01M 11/02

(52) U.S. Cl. 436/523; 422/102; 427/331; 427/372.2; 359/368

(57) ABSTRACT

Microplates containing spherical “microsphere” fluorescence standards are disclosed. The microplates can be prepared using several methods including airbrushing, application with an inkjet printer, or controlled evaporation. Spherical bead standards containing two or more regions stained with dyes of different fluorescence lifetimes, and methods for their preparation are also disclosed. The microplates can be used as calibration standards for fluorescence and confocal microscopes, and as calibration tools for microscope-based high content screening (“HCS”) instruments.

### CROSS REFERENCE TO RELATED APPLICATIONS

The present application claims priority to U.S. Provisional Patent Application Ser. No. 60/606,694 filed Sep. 1, 2004, the contents of which are incorporated herein by reference.

### FIELD OF THE INVENTION

The invention relates to microplates containing multicolor fluorescent microspheres and their use as calibration standards for microscope-based high content screening instruments.

### DESCRIPTION OF RELATED ART

High throughput screening (“HTS”) assays are becoming increasingly common in research and development laboratories. These assays allow researchers to screen a large quantity of materials in a relatively short period of time. Serial assays of each material would be prohibitively expensive and time consuming.

Conventional microplate assay systems are typically performed using cell-free liquid systems, such as ELISA immunoassays or enzyme activity assays. The resulting signal is typically detected resulting from fluorogenic or chromomeric reactions. These assays have relatively high concentrations of enzymes, high turnover numbers of substrates, and relatively high signal intensities. Each well of the microplate is viewed as an entire unit, with each well of the microplate being evaluated individually.

Cell-free systems do not effectively model the complex interactions, signaling, and feedback that are present in intact cells and tissues. Accordingly, it would be desirable to develop high throughput screening systems that use intact cells. Despite the widespread usage of microplate assay systems, they are not well suited for cellular assays. Staining or reactions in a specific cell would generate a low intensity signal, and could be incorrectly interpreted as a “negative” by the system.

High content screening (“HCS”) systems are being developed to be able to examine individual cells in a microplate well. The HCS systems can find and focus on individual cells, and perform pattern recognition and validation. The HCS systems can measure multiple fluorescent images to assay gene expression, cell-to-cell interactions, chemotaxis, and motility. The HCS systems can also measure cell cycle stages, cell morphology changes, cellular differentiation, physiological response to signals, and changes in cytoskeletal structure. The HCS systems can also be used to measure changes in spatial distribution caused by receptor trafficking and complex formation.

HCS systems are inherently complicated. They involve microplate readers, fluorescence microscopes, and image analysis. In order to obtain valuable, reproducible data, the systems must be well calibrated. Despite this need, there is no widely used or accepted calibration standard for HCS systems.

One idea for a standard would be to first culture a particular cell line in the micro-well to a preferred density, then stain the cells with multiple fluorescent dyes and fix the cells on the bottom of the wells. However, cells are relatively unstable, non-uniform, and difficult to handle in a reliable manner. The biological diversity of a cell population makes this a less useful standard, and would be a less effective calibration tool. In addition, the residual dye molecules in the wells after staining process are difficult to remove completely, leading to increased background fluorescence.

U.S. Pat. No. 5,786,219 (issued Jul. 28, 1998) described fluorescently labeled microspheres having at least one internal fluorescent spherical zone. The microspheres were described as being useful for improving the performance of an instrument that performs three-dimensional spatial analysis. A three-dimensional representation of one or more microspheres is obtained, compared with the known three-dimensional geometry of the microspheres, and the instrument is adjusted if the obtained and known geometries are different.

TetraSpeck beads have been commercially available from Molecular Probes, Inc. (Eugene, Oreg.) since 1996. These polystyrene microsphere beads have been stained throughout with a mixture of four different fluorescent dyes. TetraSpeck beads mounted on glass slides have been commercially available from Molecular Probes, Inc. (Eugene, Oreg.) since 1998.

FocalCheck beads have been commercially available from Molecular Probes, Inc. (Eugene, Oreg.) since 1996. FocalCheck beads mounted on glass slides have been commercially available from Molecular Probes, Inc. (Eugene, Oreg.) since 2000. These polystyrene microsphere beads have been stained throughout with a first dye, and stained with a second dye over the outer surface of the bead. The second dye penetrates the bead only to a limited depth, and not fully into the bead. These polystyrene microspheres can have several types of staining configurations (patterns). They have been stained throughout with a first dye, and stained with a second dye over the outer surface of the beads. The second dye penetrates the bead only to a limited depth, and not fully into the bead. When viewed in cross section in the confocal laser-scanning microscope, the resulting beads exhibit disk stain of one dye, and a ring stain of the other dye. Alternatively, they have been stained with two or three fluorescent dyes simultaneously and the dyes are allowed to penetrate to only a limited depth within the microspheres. The resulting beads have a well-defined dye layer that, when viewed in cross section in the confocal laser-scanning microscope, exhibit ring stains of two or three different fluorescent colors.

Despite advances made in developing and using HTS and HCS systems, there still exists a need for reliable, consistent standards that allow a user to calibrate the instrumentation and validate the results obtained from their use.

### SUMMARY OF THE INVENTION

Microplates containing spherical bead standards, and methods for their preparation are disclosed. Preparation of monodisperse distributions of beads in microplate wells can be achieved by various methods including airbrushing, application with an inkjet printer, or controlled evaporation. Spherical bead standards containing two or more regions stained with dyes of different fluorescence lifetimes, and methods for their preparation are also disclosed.

### DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIG. 1 shows a fluorescence lifetime measurement of a time resolved microsphere containing orange BODIPY dye and platinum porphine dye. The x-axis is time in nanoseconds; the y-axis is relative fluorescence intensity.

FIG. 2 shows the alignment of an instrument using microsphere standards.

FIG. 3 shows vertical (z-axis) optical sectioning of a microplate containing microspheres.

### DETAILED DESCRIPTION OF THE INVENTION

While compositions and methods are described in terms of “comprising” various components or steps (interpreted as meaning “including, but not limited to”), the compositions and methods can also “consist essentially of or “consist of the various components and steps, such terminology should be interpreted as defining essentially closed-member groups.

Microplates

One embodiment of the invention relates to microplates having one or more wells containing spherical standards. Microplates are typically square or rectangular in shape, having a series of rows and columns of wells that are open on the top face of the microplate. The wells are typically water-tight, and are typically used to hold a liquid and various analytes. In some microplate designs, the bottoms of the wells are made of permeable membranes, PVDF, glass fiber or other types of filter on the bottom rather than an impermeable plastic bottom. The wells are typically circular in cross-section, although other shapes such as square or triangular cross sections are possible. Microplates are sometimes referred to by other names such as microtiter plates.

The number of rows and columns of wells can generally be any number of rows and columns, but typically are 8 rows×12 columns (96 wells), 16 rows×24 columns (384 wells), or 32 rows×48 columns (1536 wells). Microplates are typically made out of plastic such as polystyrene, or glass, but could be prepared out of other materials. The spherical standards can be present in one or more individual wells, or can be present across one or more rows or columns. The microplates can be transparent, translucent, or opaque. It is presently preferred that the microplates be black in color to reduce “cross-talk” between wells. It is presently preferred that the bottom of the wells be clear, flat, and thin. The bottoms of the wells can be untreated or treated. Wells can be treated with positively charged coating reagents such as poly-lysine or poly-arginine. Wells can alternatively be treated with negatively charged coating reagents such as poly(acrylic acid).

Spherical standards, and methods for their preparation are described in co-assigned U.S. Pat. No. 5,786,219.

The spherical standards present in any particular well can be homogeneous (i.e. all spherical standards are of the same shape, size, and composition), or can be heterogeneous (i.e. a mixture of multiple different spherical standards). Each well can contain the same standards, or can contain different standards (e.g., one well can have 4 micrometer standards, and another well can contain 6 micrometer standards).

The spherical standards can generally have any diameter. Generally, the diameter can be about 1 μm to about 100 μm. Presently preferred diameters are about 4 μm to about 16 μm. Specific examples of diameters include about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 30 m, about 40 μm, about 50 μm, about 60 μm, about 70 μm, about 80 μm, about 90 μm, about 100 μm, and ranges between any two of these values.

The spherical standards can be prepared from generally any material. It is presently preferred that the spherical standards are prepared from a polymer material. Example polymer materials include polymers and copolymers of styrenes and divinyl benzenes; an acrylate or methacrylate ester; an acrylic acid or methacrylic acid; an acrylamide or methacrylamino; an acrylonitrile or methacrylonitrile; vinyl and vinylidene halides, esters and ethers; alkenes, including ethylene, propylene, butadiene and isoprene; epoxides and urethanes.

The spherical standards have at least two concentric spherical zones, an inner center zone and one or more outer concentric zones. At least one of the zones is stained with at least one fluorescent dye. In one embodiment, the inner concentric spherical zone is stained with a first fluorescent dye, and the outer concentric spherical zone is stained with a second fluorescent dye. The first and second fluorescent dyes can be same or different. If the first and second fluorescent dyes are the same, they can be present at a different concentration in the inner and outer concentric spherical zones. A fluorescence cross-section of the spherical standards can have a circular inner region corresponding to the center of the standard, and one or more outer rings corresponding to the outer concentric spherical zone or zones.

The one or more fluorescent dyes in the spherical standards can be selected to match the excitation source (e.g., a laser such as an argon-ion laser, a krypton-argon laser, or a helium-neon laser), and/or an optical filter commonly used in fluorescence microscopes or confocal laser-scanning microscopes.

The spherical standards are preferably present in the well or wells as a monolayer on the bottom of the well, i.e. a single layer of spherical standards. The monolayer is preferably even across substantially all of the bottom of the well. It is presently preferred that the standards are distributed across the bottom of the well, but not along the side of the well.

The spherical standards are preferably affixed to the bottom of the well. The affixation can be through a chemical interaction such as a covalent bond, ionic attraction, van der waals interaction, hydrophobic interaction, or other such interactions. The affixation can alternatively be through physical methods such as evaporation or glue or resin.

Time Resolved Microspheres

An additional embodiment of the invention relates to spherical standards having at least two concentric spherical zones, wherein the spherical zones comprise fluorescent dyes having different fluorescence lifetimes. The number of concentric spherical zones can be 2, 3, 4, 5, 6, or other positive integer values.

The spherical standards can generally have any diameter. Generally, the diameter can be about 1 um to about 100 um. Presently preferred diameters are about 4 μm to about 16 μm. Specific examples of diameters include about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 30 μm, about 40 μm, about 50 μm, about 60 μm, about 70 μm, about 80 μm, about 90 μm, about 100 μm, and ranges between any two of these values.

In one embodiment, the spherical standards have an first inner spherical zone containing a first fluorescent dye, and a second outer spherical zone containing a second fluorescent dye. The first fluorescent dye has a first fluorescence lifetime, and the second fluorescent dye has a second fluorescence lifetime. The first fluorescence lifetime is different from the second fluorescence lifetime. The first fluorescence lifetime can be greater than the second fluorescence lifetime, or the second fluorescence lifetime can be greater than the first fluorescence lifetime.

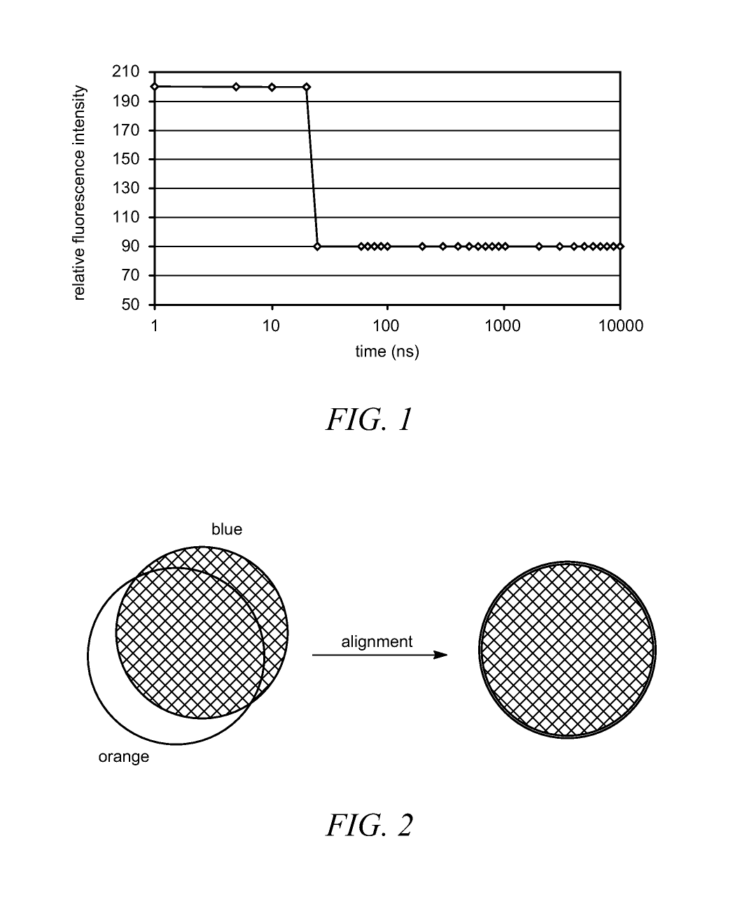

Example pairs of short and long fluorescence lifetime dyes include: red BODIPY (short, about 20 ns) and europium chelate (β-diketone) (long, about 80 μs); orange BODIPY (short, about 20 ns) and platinum porphine (long, about 300 μs). An example of a fluorescence lifetime measurement is shown in FIG. 1.

In an alternative embodiment, the spherical standards have three or more concentric spherical zones, each comprising a different fluorescent dye having a different fluorescence lifetime.

When irradiated with pulse light of an appropriate wavelength, the time resolved microspheres will fluoresce in multiple time stages. For example, for a microsphere containing two concentric spherical zones each containing a different dye, a first emission time stage will reflect emission of both dyes. After the lifetime of the shorter lifetime dye has passed, the second emission time stage will reflect emission of only the longer lifetime dye.

The intensity of the fluorescence before and after expiration of the shorter lifetime dye is a fixed value, and can be used to calibrate an instrument. If “A” is the intensity of the shorter lifetime dye in the microsphere, and “B” is the intensity of the longer lifetime dye in the microsphere, then (A+B)/B is a fixed numerical value.

The surface of the microspheres can have functional groups capable of forming covalent bonds with other molecules or materials, or can lack functional groups. If the surface lacks functional groups, then it will be relatively hydrophobic, and can interact with other surfaces by hydrophobic interactions. Carboxyl functional groups can be used for covalent coupling of amine-containing molecules using reagents such as water-soluble carbodiimide reagents (EDAC; commercially available from Molecular Probes, Inc.; Eugene, Oreg.). Amino functional groups can be coupled to a wide array of amine-reactive molecules such as succinimidyl esters and isothiocyanates. Aldehyde functional groups can be coupled to amines under relatively mild conditions. Other examples of functional groups include chloromethyl groups and amidine groups.

Methods of Preparing Microplates

Microplates having one or more wells containing spherical standards and/or time resolved microsphere standards can be prepared. Extensive experimentation by the instant inventor was required to identify effective methods of preparation.

Effective methods are preferably easy to perform, deliver reproducible amounts of spherical standards to the wells, and do not introduce undesirable background fluorescence or other properties that interfere with the use of the microplates. Effective methods also deliver well-attached microspheres to the bottom of a well (or wells) of the microplate and ensure the beads to remain fixed despite long-term storage. The microspheres preferably stay attached to the microplates for at least about 6 months of storage at room temperature, and even more preferably for at least about 12 months of storage at room temperature. The microplates are also preferably resistant to the mechanical stresses that occur during normal shipping and handling of biological research materials.

In all effective methods, the above described spherical standards and/or time resolved microsphere standards can be applied to a commercially available microplate. As described above with relation to the microplates themselves, the spherical standards present in any particular well can be homogeneous (i.e. all spherical standards are of the same shape, size, and composition), or can be heterogeneous (i.e. a mixture of multiple different spherical standards). Each well can contain the same standards, or can contain different standards (e.g., one well can have 4 micrometer standards, and another well can contain 6 micrometer standards).

Suspension of microspheres can be prepared in a solvent, or in a solvent containing a detergent (sometimes referred to as a surfactant). The presence of a detergent can aid in obtaining a monodisperse distribution of microspheres, and can aid in the attachment of microspheres to the bottom of the well or wells. Detergents can generally be non-ionic detergents (such as n-dodecyl-β-D-glucopyranoside, n-dodecyl-β-D-maltoside, n-octyl-β-D-thioglucopyranoside, NP-40, Triton-X-100, Triton X-114, Tween 20, or Tween 80), cationic detergents (such as cetyltrimethyl ammonium bromide, cetyldimethyl ammonium bromide, or tetradecyltrimethyl ammonium bromide), anionic detergents (such as sodium dodecyl sulfate or sodium lauroyl sarcosine), or zwitterionic detergents (such as 3-[(3-cholamidopropyl)dimethylammonio]-1-propanesulfonate (“CHAPS”) and lauryldimethylamine oxide).

The suspension of microspheres can also contain a linear polymer to aid in the attachment of microspheres to the bottom of the well or wells. Examples of such polymers include poly(vinyl alcohol) and polyacrylamide.

A method of preparing a standardized microplate can comprise providing a microplate comprising a plurality of wells, preparing a suspension of microspheres in a solvent, applying the suspension to one or more wells using an airbrush, and removing the solvent to afford a standardized microplate. The solvent can generally be any solvent. Presently preferred solvents comprise water, alcohol, or a mixture thereof. The solvent can further comprise a detergent, a buffer, a salt, poly(vinyl alcohol), or other materials.

The preparing the suspension step can comprise physical mixing, shaking, or soncating in a sonicator device. The removing the solvent step can generally be any method of removing, and is presently preferred to be evaporation at ambient pressure and temperature. The removing step can alternatively be performed at elevated temperature, lowered pressure, or both. The duration of the applying step can be fixed per well, or can be varied to allow for different loading of the microspheres. The distance from the microplate to the tip of the airbrush can be fixed per well, or can be varied to allow for different loading of the microspheres. The duration of the applying step can generally be any duration, and can be adjusted according to the concentration of microspheres in the suspension (e.g., a lower concentration may lead a skilled operator to select to a longer duration). The distance from the microplate to the tip of the airbrush can generally be any distance, and can be adjusted according to the concentration of microspheres in the suspension (e.g., a lower concentration may lead a skilled operator to select a shorter distance). The choice of duration and distance can be readily selected by one of ordinary skill in the art. Exemplary durations are about 0.1 seconds, about 0.2 seconds, about 0.3 seconds, about 0.4 seconds, about 0.5 seconds, about 0.6 seconds, about 0.7 seconds, about 0.8 seconds, about 0.9 seconds, about 1.0 seconds, and ranges between any two of these values. Exemplary distances are about 3 inches (about 7.6 cm), about 6 inches (about 15.2 cm), about 9 inches (about 22.9 cm), about 12 inches (about 30.5 cm), about 15 inches (about 38.1 cm), about 18 inches (about 45.7 cm), about 21 inches (about 53.3 cm), about 24 inches (about 61.0 cm), and ranges between any two of these values.

An alternative method of preparing a standardized microplate can comprise providing a microplate comprising a plurality of wells, preparing a suspension of microspheres in a solvent, loading the suspension in an inkjet printer cartridge, loading the inkjet printer cartridge into an inkjet printer, applying the suspension to one or more wells using the inkjet printer, and removing the solvent to afford a standardized microplate. The solvent can generally be any solvent. Presently preferred solvents comprise water, alcohol, or a mixture thereof. The solvent can further comprise a detergent, a buffer, a salt, poly(vinyl alcohol), a surfactant, or other materials. The preparing the suspension step can comprise physical mixing, shaking, or soncating in a sonicator device. The removing the solvent step can generally be any method of removing, and is presently preferred to be evaporation at ambient pressure and temperature. The removing step can alternatively be performed at elevated temperature, lowered pressure, or both. The duration of the applying step can be fixed per well, or can be varied to allow for different loading of the microspheres. The duration of the applying step can generally be any duration, and can be adjusted according to the concentration of microspheres in the suspension (e.g., a lower concentration may lead a skilled operator to select to a longer duration). The choice of duration can be readily selected by one of ordinary skill in the art. Exemplary durations are about 0.1 seconds, about 0.2 seconds, about 0.3 seconds, about 0.4 seconds, about 0.5 seconds, about 0.6 seconds, about 0.7 seconds, about 0.8 seconds, about 0.9 seconds, about 1.0 seconds, and ranges between any two of these values. The inkjet printer can generally be any inkjet printer capable of being indexed or controlled in three dimensions. Inkjet printers are commercially available from a wide variety of manufacturers, such as ImTech, Inc. (Corvallis, Oreg.) and Advanced Automation of California (San Clemente, Calif.).

An alternative method of preparing a standardized microplate can comprise providing a microplate comprising a plurality of wells, preparing a suspension of microspheres in a solvent, applying the suspension to one or more wells, and removing the solvent to afford a standardized microplate. The solvent can generally be any solvent. Presently preferred solvents comprise water, alcohol, or a mixture thereof. It is presently preferred that the solvent contain low concentrations of volatile alcohol solvents. High concentrations of volatile alcohol solvents can lead to high evaporation rates, and reduced acceptability of the prepared standardized microplate. The solvent can further comprise a detergent, a buffer, a salt, poly(vinyl alcohol), a surfactant, or other materials. The preparing the suspension step can comprise physical mixing, shaking, or soncating in a sonicator device. The applying step can be performed with a pipette, a micropipette, a multi-channel micropipette, a robotic liquid handling system, or other systems commonly used in biological research. The removing the solvent step can generally be any method of removing, and is presently preferred to be evaporation at ambient pressure and temperature. The removing step can comprise allowing the microspheres to settle to the bottom of the well, and removing the supernatant prior to the evaporation step. The removing step can alternatively be performed at elevated temperature, lowered pressure, or both. The volume of suspension added per well can be fixed per well, or can be varied to allow for different loading of the microspheres. The volume can generally be any volume up to the maximum volume of the well, and can be adjusted according to the concentration of microspheres in the suspension (e.g., a lower concentration may lead a skilled operator to select a larger volume). The volume can be readily selected by one of ordinary skill in the art. Exemplary durations are about 0.1 seconds, about 0.2 seconds, about 0.3 seconds, about 0.4 seconds, about 0.5 seconds, about 0.6 seconds, about 0.7 seconds, about 0.8 seconds, about 0.9 seconds, about 1.0 seconds, and ranges between any two of these values.

Methods of using Microplates

The above described microplates can be used to calibrate microscope-based instruments, and are particularly attractive for the calibration of high content screening (“HCS”) instruments. Autofocusing, object recognition, and accurate enumeration of cells are often the first steps in HCS measurements. This is especially important when the viewing field is moved from well to well. The above described microsphere coated microplate wells can simulate cultured cells for validation and calibration purposes. The calibration method can comprise providing a microscope-based instrument such as a fluorescence microscope, providing one of the above described microplates, imaging one or more wells with the microscope to obtain an image, comparing the image with a predicted image, and calibrating the microscope (if required). The calibration methods can comprise providing a microplate having one or more wells containing spherical standards and/or time resolved microsphere standards as described above, imaging the spherical standards and/or time resolved microsphere standards using a microscope-based instrument, and calibrating the instrument. The calibrating step can include correcting auto-positioning, correcting auto-focusing, correcting enumeration, correcting laser alignment, correcting chromatic aberration, correcting axial resolution, monitoring the stability and performance of lamp sources such as mercury and xenon lamps, or combinations thereof. The exact method of making these corrections will vary somewhat depending on the particular microscope-based instrument used, but are all within the abilities of the skilled artisan.

The above described microplates can be used to correct for chromatic aberration. High performance objective lenses are designed to give no apparent variation in color across the field of view when examining biological specimens under white light. However, the pure colors used for laser excitation are much more sensitive to slight errors in chromatic correction, and separation of the fluorescence images into several channels can produce misregistration, especially in the multiple pinhole confocal microscope in a HCS instruments.

Since there can be two or more dye combinations in one microsphere with special staining patterns, it can be used to test each objective lens for chromatic aberration by simply acquiring an X-Y image across the widest available field of view, and then comparing the bead images (location and intensity) in each channel. Their ring/disc patterns are more visually striking and present a larger target than submicron beads for testing with a line query. The large beads (6 μm or 15 μm diameter) are easy to locate, and it requires less dexterity with the mouse to accurately bisect the bead image with a line query. Colocalization plots can be used on a single FocalCheck bead to present the accuracy of color registration, and to chart improvement as a procedure is carried out.

A method to correct for chromatic aberration can therefore comprise providing a fluorescence microscope, providing one of the above described microplates, obtaining an X-Y image across the widest available field of view, comparing bead images in the X-Y image, and calibrating the microscope (if required).

An additional embodiment involves the co-alignment of a laser scanning system. The above described microspheres are isotropic—the ring/disc staining pattern is independent to the orientation of an illumination laser beam. The microspheres can be used to check the alignment of a confocal laser scanning system. The method can comprise providing a confocal laser scanning system, providing one or more of the above described microspheres or one of the above described microplates, imaging the microspheres in multiple dimensions to obtain multiple images, and calibrating the system such that the multiple images are coincident.

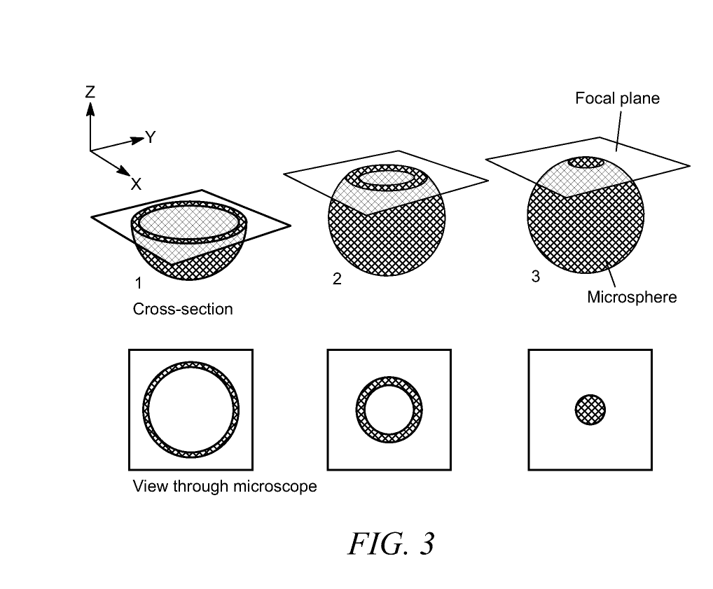

An additional embodiment is directed towards methods for determining Z-axis resolution that can be resolved with a confocal microscope in a HCS system. A serial optical sectioning of microspheres from the top to the center then to the bottom along the Z-axis reveal a continuous pattern of disc-to-ring-to-disc images. For example, in the case when a 15 μm-diameter bead coated microplate is used, as the focal plane of the confocal microscope sweeps down, the microspheres first appear as a solid disc before the ring shape emerges and enlarges to final 15 μm-diameter ring, then diminishes and finally disappear. By measuring the diameter of the ring and disc, the “optical sectioning performance” of a HCS system under different conditions of aperture size and objective lens selection can be evaluated. If needed, corrections to the microscope can then be made accordingly (FIG. 3).

An additional embodiment is directed towards methods for determining the flatness of an individual well or the whole plate. In a HCS system, a whole microplate instead of regular glass slide is scanned. The plate drive moves the whole plate along X-Y and X-Z axis so each well can be examined; or alternatively, the plate is in a fixed position while the microscope is moved with respect to the plate. In either case, the relative position between microscope objective lens and the plate is constantly changing. To obtain reliable microscopic images, it is especially desirable to check the flatness of the well to be examined (and the performance of the whole system). To determine the flatness, a X-Z image of a group microsphere beads attached on the bottom of the well is collected. In a normal case, these image of the beads should: 1) be spherical and always appear circular, and 2) the diameter of all the beads shown in the image should be the same which indicating they are all on the same focal plane). If the diameter of the beads in the left side of the image, for example, appear smaller (or larger) then those on the right side, this would be an indication of a tilting bottom of the microplate.

An additional embodiment is directed towards methods for the co-alignment of excitation beams. By taking a multi-channel X-Z image of a microsphere bead, the co-alignment of laser lines of a confocal microscope can be confirmed. This is especially desirable for UV-light excited confocal microscope because even apo-chromatic lenses require extra correction to bring the plane of focus of the UV and visible beams to exactly the same point within the specimen. Slight errors in co-alignment will appear as slight vertical shift between channels, producing single-color figures above and below the circular image of each bead. Standard X-Y images will show any lateral alignment errors between excitation lines, and a Z-series will show up both types of error in a single data set.

An additional embodiment is directed towards methods for the evaluation and monitoring the stability and performance of Mercury and Xenon lamp sources. Given that fluorescence microspheres coated on the bottom of the plate wells are a relatively stable and consistent fluorescence source, a researcher can select at random a field of beads and acquire a first image. The instrument parameters can be recorded for future use. The intensities of the beads and background can be determined using standard analysis tools and that data recorded into a log file for future reference. At some later point in time the same beads could be used, using the same instrument parameters to acquire a second image set. The intensities of the beads can be determined and compared to the results of previous measurements. Statistical analysis can be used to determine if the beads intensities are significantly different from previous measurements. Should this be the case it would indicate some change in the performance of the optical system of the instrument, most likely a drop in the output of the lamp source. Alternatively time-lapse imaging of a single field of beads can also be used to monitor the stability of a lamp that may be giving inconsistent results. Plotting the intensity overtime would reveal any unusual fluctuations in signal of the beads that would be attributable to fluctuations in the lamp intensity.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### Examples

### Example 1

### Preparation of Microspheres

Microspheres having concentric fluorescent spherical zones can be prepared using the methods described in U.S. Pat. No. 5,786,219 (issued Jul. 28, 1998). The microspheres have at least two distinct concentric zones, labeled with one or more fluorescent dyes.

FocalCheck microspheres of various micrometer diameters (e.g., 1, 6, and 15 micrometers) are commercially available from Molecular Probes, Inc. (Eugene, Oreg.).

### Example 2

### Preparation of Time-Resolved Microspheres

Microspheres having a uniform orange stain (with short fluorescence life time, about 18 ns) and a deep red ring stain (with long fluorescence life time, about 300 μs) were prepared according to the following method.

The following stock solutions were prepared: 4,4-difluoro-1,3-diphenyl-5,7-dipropyl-4-bora-3a,4a-diaza-s-indacene (5.0 mg; Molecular Probes Inc.; Eugene, Oreg.) was dissolved in methylene chloride to give a stock solution having a concentration of 2.0 mg/mL (Stock solution A). Pt (II) Meso-tetra (pentafluorophenyl) porphine (4.0 mg, Frontier Scientific, Inc., Logan, Utah) was dissolved in methylene chloride to give a stock solution having a concentration of 4.0 mg/mL (Stock solution B).

A 1.0 mL suspension (10% solids) of 15.0 μm microspheres (polystyrene/2% divinylbenzene; Bangs Laboratories) was placed in a test tube. Approximately 3 mL of ethanol was added to the test tube, and the microspheres were resuspended.

The uniform staining solution was prepared by combining 150 μL of stock solution A, 850 μL of methylene chloride, and 1.0 mL of ethanol. The uniform staining solution was added to the stirring microsphere suspension, and the suspension was stirred for 15 minutes. The suspension was then quickly centrifuged for 5 seconds, and the supernatant solution was discarded. The microspheres were then washed twice, with methanol using sonication of the suspension during the second wash step. The resulting microsphere pellet was then washed three more times using a 0.02% solution of TWEEN-20 with an increase in the centrifugation time to 1 minute and using sonication of the suspension during the first and third wash step. The uniformly stained microspheres were centrifuged, followed by addition of 3 mL of ethanol. The mixture was stirred well to resuspend the microspheres.

A ring staining solution was prepared by combining and thoroughly mixing 100 μL of stock solution B, 450 μL of ethanol, and 35 μL of methylene chloride. The staining solution was added to the stirring microsphere suspension, and the microsphere suspension was stirred for exactly 1 minute. The suspension was then quickly centrifuged for 5 seconds and the supernatant solution was discarded. The microspheres were then washed three times, as above, using methanol and with sonication of the suspension during the second wash step. The resulting microsphere pellet was washed three times using a 0.02% solution of TWEEN-20 (VWR Scientific; West Chester, Pa.), centrifuging for 1 minute in each step and with sonication of the suspension during the first and third wash. After washing was complete, the microspheres were suspended in approximately 5 mL of 0.02% TWEEN-20, carefully vacuum filtered using a polyester filter, and washed with additional 0.02% TWEEN-20. The stained microspheres were then resuspended in 0.02% TWEEN-20 to the desired suspension concentration.

The resulting microspheres possess a well-defined region of shallow deep red fluorescent staining and uniform orange fluorescence throughout the microsphere. When viewed in cross-section under a Zeiss LSM 510 confocal microscope they display orange fluorescent interiors and fluorescent deep red ring staining (with longer fluorescence lifetime).

### Example 3

### Determining Acceptability of Microspheres Coated onto Microplates

Microplates coated with microspheres were examined using a Zeiss Axioplan fluorescence microscope to assay the coating acceptability. Microscope objectives and stages were thoroughly cleaned to prevent the transfer of any oil or dirt to the microplates. The plates were handled with cotton-gloved hands, touching only the edges of the plate. The plates were examined using proper objective and fluorescence excitation/emission filter combinations (such as UV, FITC, PE or Cy5).

The plates were examined for the presence or absence of the following three desirable qualities. First, all of the beads should be firmly attached to the bottom of the micro-wells (i.e. no free moving microspheres were observed) and be at same focal plane. Second, there should be several hundreds to thousands of beads distributed throughout the well. The distribution should be random and even. Ideally, more than 95% of the beads are mono-disbursed. Aggregated beads may be present, but the overall degree of aggregation in the well preferably is less than 5%. Third, no background fluorescence from the plate, coating ingredients such as detergents, linear polymers or other material used for coating should be observed.

### Example 4

### Coating of Microspheres onto Polymer Microplate using a Rotary Vacuum Device

200 μL of 6 μm diameter FocalCheck microspheres stock (prepared according to Example 1) was resuspended in 10 mL of solution containing 0.05% Tween 20 surfactant (VWR Scientific, West Chester, Pa.). The bead suspension was sonicated in a Branson 1510 water bath sonicator for 5 minutes at room temperature to promote mono-disbursement of the beads.

100 μL of the FocalCheck bead suspension was transferred to each well of a 96 MICROWELL cell culture plate microplate (polymer base, Nunc Life Science Products, Rochester N.Y.; MICROWELL is a registered trademark of Nunc Corporation). The beads were allowed to settle for two hours.

The microplate was placed in a Thermo Savant SPEEDVAC centrifuge model SC250 DDA (Thermo Electron Corp.; Waltham, Mass.; SPEEDVAC is a registered trademark of Thermo Savant, Inc.). The centrifuge was run at a speed of 1,200 rpm, and at a “high” drying rate for 4 hours to evaporate the liquid.

Upon drying, the wells were evaluated. Microscopic examination indicated that more than 60% of the beads were accumulated along the edge of the wells, or form several concentric circles (“rings”), rather than being in a monodisperse distribution along the bottom of the wells. This result was unacceptable.

### Example 5

### Coating of Microspheres onto Polystyrene Microplate using a Rotary Vacuum Device

200 μL of 6 μm diameter FocalCheck microspheres stock (prepared according to Example 1) was resuspended in 2.5 mL of a solution containing 0.05% Tween 20 surfactant (VWR Scientific; West Chester, Pa.). The bead suspension was sonicated in a Branson 1510 water bath sonicator for 5 minutes at room temperature to promote mono-disbursement of the beads. 2.5 mL of ethyl alcohol (200 proof, AAPER Alcohol and Chemical Corp., Shelbyville, Ky.) was added to the bead suspension and mixed well.

50 μL of the FocalCheck bead suspension was transferred to each well of a CellBIND 96 well flat clear bottom black polystyrene microplate (Catalog No. 3340; Corning; Life Sciences; Corning, N.Y.; CELLBIND is a registered trademark of Coming, Inc.). The beads were allowed to settle for two hours.

The bead distribution obtained was very similar to that obtained in the previous example, and was similarly unacceptable.

### Example 6

### Coating of Microspheres using a Rotary Vacuum Device with Surfactant and PVA

100 μL of 6 μm FocalCheck microspheres stock (prepared according to Example 1) was resuspended in 10 mL of solution containing 0.05% Tween 20 surfactant, 0.1% poly (vinyl alcohol) (87-89% hydrolyzed, average MW: 124,000-186,000; Sigma-Aldrich Corp.; St. Louis, Mo.), and 20% ethyl alcohol (200 proof, AAPER Alcohol and Chemical Corp., Shelbyville, Ky.). The beads suspension was sonicated in a water bath sonicator for 5 minutes at room temperature to obtain mono-disbursement of the beads.

100 μL of the FocalCheck bead suspension was transferred to each well of a 96 MICROWELL cell culture plate microplate (polymer base, Nunc Life Science Products; Rochester, N.Y.). The beads were allowed to settle for 3 to 4 hours.

The plate was placed in a Thermo Savant SPEEDVAC centrifuge model SC250 DDA. The centrifuge was run at a speed of 1,200 rpm, and a “medium” drying rate for 4 hours to evaporate the liquid.

The evaporation rate of water and the different sedimentation forces applied to wells in different locations of the microplate resulted in an unacceptable bead distribution.

### Example 7

### Coating of Microspheres onto Micro-Well Plates using a Rotary Vacuum Device at High Speed and Temperature

FocalCheck microspheres were transferred to 96 well micro-well plates as described in the previous example. The plate was placed in a Thermo Savant SPEEDVAC centrifuge model SC250 DDA. The centrifuge was run at a speed of 1,800 rpm, and a “high” drying rate for 2 hours to evaporate the liquid.

The bead distribution in the microplate was unacceptable due to the same factors encountered in the previous example.

### Example 8

### Coating of Microspheres onto Micro-Well Plates using an Air Brush

2 mL of FocalCheck microspheres stock as prepared in Example 1 was mixed with 2 mL of 0.05% Tween 20 (VWR Scientific; West Chester, Pa.), 0.1% poly (vinyl alcohol) (87-89% hydrolyzed, average MW: 124,000-186,000). The mixture was sonicated for three minutes. The bead suspension was transferred to a fluid bottle of a H & HS single action, external-mix airbrush with a 15 mm fluid nozzle. The airbrush was attached through an air hose to a piston-type Airmaster Plus™ air compressor (Kopykake Enterprises, Torrance, Calif.) that can provide a constant airflow. Four 96 well black micro-well plate with clear polystyrene bottom, poly-D-lysine treated (Corning Inc. Corning, N.Y.) were placed inside a Labconco fume hood (Labconco Corp., Kansas City, Mich.).

A plastic rectangular grid was placed on the top face of the microplate. The grid had a similar dimension as the plate, 96 holes positioned above the wells of the microplate, and holes that were about ⅔ of the diameter of the wells. The grid was used to focus the airflow and subsequently direct the beads to the bottom of the well; and to deflect the less desirable beads /air flow.

The control valve of the air compressor was adjusted to select a desired airflow rate. The FocalCheck microspheres were sprayed directly on to the bottom of each well. A constant distance of about 9 inches (22.9 cm) was maintained between the tip of the brush and the plate. A spray time of 0.5 seconds per well was used.

After air brushing, the plates were kept inside of the fume hood for an additional 30 minutes to allow the minimal amount of aqueous medium evaporate at room temperature.

Microscopic examination indicated that the bead distribution is good with firm attachment and low background fluorescence. Reproducibility may be improved by using a robotics or a mounted airbrush positioned above a conveyor belt that passes microplates beneath the brush at a fixed rate.

### Example 9

### Coating of Microspheres onto Micro-Well Plates using an Inkjet Printer

2 mL of 4 micrometer FocalCheck microspheres stock prepared in Example 1 was mixed with 2 mL of 0.1% Tween 20 (VWR Scientific; West Chester, Pa.), 0.2% poly (vinyl alcohol) (87-89% hydrolyzed, average MW: 124,000-186,000) (Aldrich Corp.; St. Louis, Mo.). The mixture was sonicated for five minutes in a water bath sonicator. 4 mL of the FocalCheck microspheres suspension was transferred to an ink cartridge attached to an IIS-56S/300S xyz Inkjet printing system (ImTech, Inc., Corvallis, Oreg.). The inkjet printer printed (sprayed) microspheres onto the bottom of wells of BD Falcon™, black, 96 well glass bottom imaging plates (BD Bioscience, Bedford, Mass.) to a predefined pattern.

The minimal amount of aqueous medium was allowed to evaporate at room temperature over 20 minutes. Examination of the microplate with a microscope revealed a favorable distribution of microspheres along the bottom of the wells.

### Example 10

### Coating of Microspheres onto Micro-Well Plates using an Inkjet Printer

2 mL of 4 micrometer FocalCheck microspheres stock prepared in Example 1 was mixed with 2 mL of 0.05% cetyltrimethyl ammonium bromide (Calbiochem Corp., San Diego, Calif.), 0.1% polyethylenimine (Polymer Enterprises Inc., Park Ridge, N.J.). The mixture was sonicated for five minutes in a water bath sonicator. 4 mL of the FocalCheck microspheres suspension was transferred to an ink cartridge following the same procedure as mentioned in the previous example. A Costar® 96 well black plates with clear bottom (polymer base, Corning Life Science, Corning, N.Y.) was used.

The minimal amount of aqueous medium was allowed to evaporate at room temperature over 20 minutes. Examination of the microplate with a microscope revealed a favorable distribution of microspheres along the bottom of the wells. The results obtained in this example were superior to those obtained in the previous example, perhaps due to the addition of cationic detergent.

### Example 11

### Coating of Microspheres onto Micro-Well Plates using Rapid Evaporation

500 μL of FocalCheck microspheres stock as described in Example 1 was resuspended in 1 mL of solution containing 0.05% Tween 20 surfactant (VWR Scientific, West Chester, Pa.). The bead suspension was sonicated in a Branson 1510 water bath sonicator for 5 minutes at room temperature to promote mono-disbursement of the beads. 9 mL of ethyl alcohol (200 proof, AAPER Alcohol and Chemical Corp., Shelbyville, Ky.) was added to the bead suspension and mixed thoroughly by votexing. The concentration of ethyl alcohol in the final suspension was quite high, about 86%.

50 μof the FocalCheck bead suspension was transferred using a multi-channel micropipette to each well of a 96 well black micro-well plate with clear polystyrene bottom (Corning Inc. Corning, N.Y.). Alternatively, the suspension can be added using a robotic liquid handling system such as the Biomek 200 Robotics System (Beckman Coulter; Fullerton, Calif.). The plate was placed in a fume hood overnight at room temperature to evaporate the aqueous and alcohol medium.

Upon drying, the microplate was examined using a Zeiss Axioplan fluorescence microscope. More than 70% of the beads were aggregated and accumulated along the edge of the wells, or formed irregular “ring” patterns rather than evenly distributed and mono-disbursed. The result was not acceptable.

### Example 12

### Coating of Microspheres onto Micro-Well Plates using Evaporation

800 μL of FocalCheck microspheres stock as described in Example 1 was resuspended in 10 mL of solution containing 0.02% SDS (VWR Scientific; West Chester, Pa.), 0.1% poly (vinyl alcohol) (87-89% hydrolyzed, average MW: 124,000-186,000) (Aldrich. Corp.; St. Louis, Mo.) to a final concentration of 1×10⁴ beads/mL. The beads suspension was sonicated in a water bath sonicator for 5 minutes at room temperature to ensure mono-disbursement of the beads.

100 μL of FocalCheck bead suspension was transferred using a multi-channel micropipette to each well of a 96 well black micro-well plate with clear polystyrene bottom, poly-D-lysine treated (Corning Inc. Corning, N.Y.). Alternatively, the suspension can be added using a robotic liquid handling system such as the Biomek 200 Robotics System (Beckman Coulter; Fullerton, Calif.). The beads were allowed to settle for 2 to 4 hours.

The supernatant was removed by vacuum through a glass Pasteur pipette. The plate was placed in a fume hood overnight to evaporate the remaining aqueous medium. The microplate was examined using a Zeiss Axioplan fluorescence microscope. The bead distribution was improved, likely due to the use of linear polymer PVA. Some areas of the microplate showed higher uneven bead accumulation.

### Example 13

### Coating of Microspheres onto Micro-Well Plates using Evaporation

800 μL of FocalCheck microspheres stock as described in Example 1 was resuspended in 10 mL of solution containing 0.02% SDS (VWR Scientific; West Chester, Pa.), 0.1% poly (vinyl alcohol) (87-89% hydrolyzed, average MW: 124,000-186,000) (Aldrich Corp.; St. Louis, Mo.) to a final concentration of 1×10⁴ beads/mL. The bead suspension was sonicated in a water bath sonicator for 5 minutes at room temperature to ensure mono-disbursement of the beads.

100 μL of FocalCheck bead suspension was transferred to each well of a 96 well black micro-well plate with clear polystyrene bottom, poly-D-lysine treated (Corning Inc. Corning, N.Y.). The beads were allowed to settle for 2 to 4 hours.

The supernatant was removed by vacuum through a glass Pasteur pipette. The plate was placed in a fume hood overnight to evaporate the remaining aqueous medium. Microscopic examination of the microplate indicated a very good bead distribution pattern. This result was superior to that obtained in the previous example, perhaps due to the presence of PVA, and an interaction between the negatively charged microspheres (due to the SDS) and the positively charged wells (due to the poly-D-lysine coating).

### Example 14

### Coating of Microspheres onto Micro-Well Plates using Epoxy Resin

Moisture insensitive Epoxy (product #250 from Epoxy Systems, Inc. Orlando, Fla.) components “A” and “B” were stirred thoroughly before mixing. 30 mL of “A” and 15 mL of “B” were combined and mixed for 2 minutes in a 100 mL glass beaker using a glass rod. 200 μL stock of the 6 μm FocalCheck beads were added to the mixture, and stirred for an additional two minutes. A 100 μL aliquot of this mixture was transferred to each well of a 96 MICROWELL cell culture plate (polymer base, Nunc Life Science Products, Rochester N.Y.) microplate (different amount of suspension can be transferred to different wells if desired). The plate was placed in a fume hood for 2 hours.

Three problems were observed with the produced plates. First, it was difficult to control the volume of mixture transferred to each well, as the viscosity of the mixture was quite high. Second, rapid curing of the epoxy caused pores in the surface of the set resin in more than half of the wells. Finally, more than 30% of the FocalCheck beads were not attached to the bottom of the wells.

### Example 15

### Coating of Microspheres onto Micro-Well Plates using Epoxy Resin

Moisture insensitive Epoxy (product #250 from Epoxy Systems, Inc. Orlando, Fla.) components “A” and “B” were stirred thoroughly before mixing. 30 mL of “A” and 10 mL of “B” were combined and mixed for 2 minutes in a 100 mL glass beaker using a glass rod. 200 μL stock of the 6 μm FocalCheck beads were added to the mixture, and stirred for an additional two minutes. A 100 μL aliquot of this mixture was transferred to each well of a 96 MICROWELL cell culture plate (polymer base, Nunc Life Science Products, Rochester N.Y.) microplate (different amount of suspension can be transferred to different wells if desired). The plate was placed in a fume hood for 2 hours.

This example differs from the previous example by using less volume of epoxy “B”. Two problems were observed with the produced plates. First, it was difficult to control the volume of mixture transferred to each well, as the viscosity of the mixture was quite high. Second, even though the cure time was increased due to the lower amount of epoxy “B” used, more than 20% of the wells were observed to have a porous surface.

### Example 16

### Coating of Microspheres onto Micro-Well Plates using Slow Curing Epoxy Resin

20 mL of Part 1 and 20 mL of Part 2 of Slow-Cure™ (30 min) Epoxy (Bob Smith Industries, Inc., Atascadero, Calif.) were mixed thoroughly for 2 minutes in a 100 mL glass beaker using a glass rod. 200 μL stock of the 6 μm FocalCheck beads (Example 1) were added to the mixture, and stirred for an additional two minutes. A 100 ρL aliquot of this mixture was transferred to each well of a 96 MICROWELL cell culture plate (polymer base, Nunc Life Science Products, Rochester N.Y.) microplate (different amount of suspension can be transferred to different wells if desired). The plate was placed in a fume hood for 2 hours.

As in the previous examples, the viscosity of the epoxy resin made it difficult to accurately dispense equal amounts of the suspension into each well.

### Example 17

### Coating of Microspheres onto Micro-Well Plates using Polyurethane Emulsion

10 mL of water-based clear Perm Enamel glaze (Delta Technical Coltings, Inc. Whittier, Calif.) polyurethane emulsion polymer and 30 mL of deionized water were mixed. The water was added to reduce the viscosity of the emulsion. 200 μL stock of 6 μm FocalCheck beads were added to the diluted polyurethane. The bead suspension was stirred slowly to avoid bubble formation for 10 minutes. A 100 microliter aliquot of the mixture was transferred to each well of a 96 well Costar® black plate with clear bottom micro-well plate (polymer base, Corning Life Science, Corning, N.Y.). The plate was placed in a fume hood for 48 hours.

A thin and firm coating was formed in each well. Unfortunately, the coating displayed a blue color auto fluorescence background when examined by fluorescence microscopy with UV excitation.

### Example 18

### Coating of Microspheres onto Micro-Well Plates using Acrylic Polymer Emulsion

200 μL stock of 6 μm FocalCheck beads was transferred to 40 mL of Liquitex high gloss varnish, a low viscosity, water-based acrylic emulsion (Dick Blick Art Material, Galesburg, Ill.). The bead suspension was stirred slowly to avoid bubble formation for 10 minutes. A 100 microliter aliquot of the mixture was transferred to each well of a 96 well Costar® black plates with clear bottom micro-well plate (polymer base, Corning Life Science, Corning, N.Y.). The plate was placed in a fume hood for 48 hours.

A thin and firm coating of acrylic polymer was formed in each well that held the FocalCheck beads tightly on the bottom of the well. Unfortunately, the coating displayed a dim blue color auto fluorescence background when examined by fluorescence microscopy with UV excitation.

### Example 19

### Use of Microplates to Validate and Calibrate Instruments

Many high content screening (HCS) systems use fully automated plate stacking modules with pick-and-place capabilities. Some modules also have microplate rotational grippers and 345 degree axis of rotation capacity. Due to these complex systems, calibration and optimization is needed.

A microplate standard having microspheres in one or more wells can be positioned in a microplate holder, and can be moved into the examining position of the instrument. Autofocusing can be visually assessed by making sure that the microspheres at the bottom of the well are focused. At least eight to ten wells can be screened. If the beads are out of focus, the autofocusing can be adjusted.

Each well of the plate containing fixed number of beads, can be used to check for accurate enumeration. Repeat reading of same well (or a row or a column of wells) should give bead count within limited variations.

If the HCS instrument contains a multiple laser confocal microscope, this can be calibrated and validated as well. This is especially desirable for UV-light excited confocal laser scanning systems, as apo-chromatic lenses require extra correction to bring the plane of focus of the UV and visible beams to the same point within the specimen. The microspheres can indicate correct image registration when the multiple ring images or ring and disc images are perfectly coincident in all dimensions. Image distortions and mismatching caused by misalignment of the lasers can be detected and corrected.

### Example 20

### Use of Microplates for Chromatic Aberration Correction

The pure colors used for laser excitation are much more sensitive to slight errors in chromatic correction than are other excitation sources. The microplates with microsphere standards are useful tools for characterizing chromatic aberrations, as their ring and disc patterns are visually striking and present a larger target for a line query. Colocalization scattergram plots can be used on a single bead to show the accuracy of color registration, and to monitor improvements due to the correction process.

### Example 21

### Use of Microplates for Axial Resolution Determinations

The microplates with microsphere standards can be used to determine the minimum separation that can be resolved with a confocal microscope in a HCS system. This measurement is useful for establishing the “optical sectioning performance” under different conditions of aperture size and objective lens selection.

As the focal plane of the confocal microscope sweeps down, the microspheres first appear as a solid disc before the ring shape emerges. The ring enlarges to a final diameter, then diminishes and finally disappears. By measuring the diameter of the ring and disc, the “optical sectioning performance” can be evaluated. If necessary, changes to the microscope can be made.

### Example 22

### Use of Microplates for Monitoring the Performance of Lamp Sources

Microplates with microsphere standards are a relatively stable fluorescence source. A researcher can record an image, and can compare later images with the original standard image. Statistical analysis can be used to determine if the bead intensity is significantly different from previous measurements. A reduction in intensity indicates a change in the performance of the optical system, most likely a reduction in the output of the lamp source.

### Example 23

### Stability of Microplates

Successfully prepared microplates exhibit long term stability and resistance to shipping and handling. The stability is preferably at least about 6 months, and more preferably at least about 12 months at room temperature. “Drop tests” of prepared microplates were performed at a vertical distance of 32 inches (81 cm). After 8 consecutive drops, all of the bound microspheres remained attached to the microplate.

### Example 24

### Packaging of Microplates

Successfully prepared microplates can be sealed across their top face using pressure-sensitive tape, or other coatings (such as a shrink wrap coating). The sealed unit can be covered with a lid and taped shut. The lid and tape can be black in color to prevent exposure to visible light over long periods of storage. The sealed unit can be used directly for instrument calibration without removing the top face coating.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the invention.

What is claimed is:

1. A microplate comprising a plurality of wells, wherein at least one of the wells contains one or more spherical standards.

2. The microplate of claim 1, wherein the spherical standards have diameters of about 1 μm to about 100 μm.

3. The microplate of claim 1, wherein the spherical standards have diameters of about 4 μm to about 16 μm.

4. The microplate of claim 1, wherein the spherical standards are affixed to the bottom of the wells.

5. The microplate of claim 1, wherein the spherical standards are arranged in a monolayer on the bottom of the wells.

6. The microplate of claim 1, wherein the spherical standards are affixed to the bottom of the wells by a covalent bond, an ionic attraction, van der waals interaction, or a hydrophobic interaction.

7. The microplate of claim 1, wherein the spherical standards are affixed to the bottom of the wells by glue or by a resin.

8. The microplate of claim 1, wherein the spherical standards comprise a polymer material.

9. The microplate of claim 1, wherein the spherical standards comprise an inner concentric spherical zone and at least one outer concentric spherical zone.

10. The microplate of claim 1, wherein the spherical standards have one inner concentric spherical zone and one outer concentric spherical zone.

11. The microplate of claim 9, wherein the inner concentric spherical zone is stained with at least one fluorescent dye.

12. The microplate of claim 9, wherein the outer concentric spherical zone is stained with at least one fluorescent dye.

13. The microplate of claim 9, wherein:
the inner concentric spherical zone is stained with a first fluorescent dye; and
the outer concentric spherical zone is stained with a second fluorescent dye.

14. A method of preparing a standardized microplate, the method comprising:
providing a microplate having a plurality of wells;
providing a suspension of microspheres in a solvent;
applying the suspension to one or more wells; and
removing the solvent to provide a standardized microplate.

15. The method of claim 14, wherein the solvent comprises water, an alcohol, and mixtures thereof.

16. The method of claim 14, wherein the suspension further comprises a detergent.

17. The method of claim 14, wherein the suspension further comprises a linear polymer.

18. The method of claim 14, wherein the suspension further comprises poly(vinyl alcohol) or polyacrylamide.

19. The method of claim 14, wherein the microspheres remain attached to the one or more wells for at least about six months at room temperature.

20. The method of claim 14, wherein the microspheres are present in the one or more wells as a monolayer.

21. The method of claim 14, wherein the applying step comprises using an airbrush.

22. The method of claim 14, wherein the applying step comprises using an inkjet printer.

23. The method of claim 14, wherein the removing step comprises allowing the solvent to evaporate at room temperature and pressure.

24. A method of calibrating a microscope-based instrument, the method comprising:
providing a microplate comprising a plurality of wells, wherein at least one of the wells contains one or more spherical standards;
imaging at least one of the wells using a microscope-based instrument to obtain a calibration image; and
calibrating the instrument.

25. The method of claim 24, wherein the instrument is a fluorescence microscope or a confocal microscope.

26. The method of claim 24, further comprising comparing the calibration image with a predicted image prior to the calibrating step.

27. The method of claim 24, wherein the calibrating step comprises correcting auto-positioning of the instrument, correcting auto-focusing of the instrument, correcting enumeration of the instrument, correcting laser alignment of the instrument, correcting chromatic aberration of the instrument, correcting axial resolution of the instrument, monitoring the stability and performance of a lamp source, or combinations thereof

28. A time resolved microsphere comprising a first concentric spherical zone and a second concentric spherical zone, wherein:
the first concentric spherical zone is stained with a first fluorescent dye having a first fluorescence lifetime;
the second concentric spherical zone is stained with a second fluorescent dye having a second fluorescence lifetime; and
the first fluorescence lifetime and the second fluorescence lifetime are different.

29. The microsphere of claim 28, wherein the microsphere has a diameter of about 4 μm to about 16 μm.

30. The microsphere of claim 28, wherein the first fluorescence lifetime is greater than the second fluorescence lifetime.

31. The microsphere of claim 28, wherein the second fluorescence lifetime is greater than the first fluorescence lifetime.

32. The microsphere of claim 28, wherein the first fluorescent dye is red BODIPY and the second fluorescent dye is europium chelate.

33. The microsphere of claim 28, wherein the first fluorescent dye is orange BODIPY and the second fluorescent dye is platinum porphine.

34. The microsphere of claim 28, further comprising a third concentric spherical zone stained with a third fluorescent dye having a third fluorescence lifetime

35. The microsphere of claim 28, further comprising a functional group on the surface of the microsphere.

36. The microsphere of claim 28, further comprising a carboxyl group, an amino group, an aldehyde group, a carbodiimide group, a succinimidyl ester group, an isothiocyanate group, a chloromethyl group, or an amidine group on the surface of the microsphere.
